(19) United States
(12) Patent Application Publication
(10) Pub. No.: US 2011/0251510 A1
(43) Pub. Date: Oct. 13, 2011

(54) RESPIRATION SENSOR FOR AN INFANT FEEDING PERFORMANCE MEASUREMENT DEVICE

(75) Inventor: Jay N. Zemel, Jenkintown, PA (US)

(73) Assignee: The Trustees Of The University of Pennsylvania, Philadelphia, PA (US)

(21) Appl. No.: 13/074,294

(22) Filed: Mar. 29, 2011

Related U.S. Application Data
(60) Provisional application No. 61/321,673, filed on Apr. 7, 2010.

(51) Int. Cl. A61B 5/08

(52) U.S. Cl. 600/537; 600/529

(57) ABSTRACT

Devices, systems and methods for measuring infant feeding performance. The device includes a body portion, a respiration sensing device for receiving respiration and an integrated circuit disposed in the body portion and electrically connected to the respiration sensing device. The body portion has a first end for receiving a fluid, a second end for passing fluid to a feeding nipple, and a conduit in fluid communication with the first end and the second end. The respiration sensing device is mechanically coupled to the body portion and is mateable with the feeding nipple. The respiration sensing device generates a signal representing a variation in temperature or airflow of the respiration during a feeding session. The integrated circuit receives the temperature or airflow signal determines a respiration pattern over the feeding session indicative of the infant feeding performance.

The subject application claims priority from U.S. Provisional Application No. 61/321,673 filed Apr. 7, 2010, the contents of which are incorporated herein by reference.

### FIELD OF THE INVENTION

This present invention relates to measurement of a feeding performance of an infant. More particularly, the present invention relates to devices and systems for measuring an infant's feeding responsiveness based on a respiration pattern and a sucking response induced by the infant on a feeding apparatus.

### BACKGROUND OF THE INVENTION

The ability to feed successfully is critical for a newborn's early development. Neonatal feeding assessment is especially important for infants born prematurely or with other medical conditions that may limit the ability to breathe, suck and swallow. Devices for assessing infant feeding performance by quantitatively measuring certain aspects of infant feeding are known. For example, a negative sucking pressure of an infant may be measured to determine if the infant has a relatively poor sucking ability. Other quantities that may be measured include, for example, an expression pressure on a nipple or a swallowing capacity.

### SUMMARY OF THE INVENTION

A method of measuring infant feeding performance. The method includes passing fluid to a feeding nipple through a conduit, passing respiration across a respiration sensor mechanically coupled to the feeding nipple, monitoring a pressure of the fluid passing through the conduit during a feeding session in the conduit, monitoring the respiration across the respiration sensor during the feeding session, and determining at least one of a sucking response from the monitored pressure or a respiration pattern from the monitored respiration over the feeding session indicative of the infant feeding performance.

According to an aspect of the invention, the temperature of inhaled and exhaled air as it flows over the respiration sensor may be used to monitor the respiration pattern relative to the sucking response.

According to another aspect of the invention, the airflow of inhaled and exhaled air as it flows over the respiration sensor may be used to monitor the respiration pattern relative to the sucking response.

According to another aspect of the invention, time characteristics of both the sucking response and the respiration pattern may be measured, for example, to determine any correlation between the respiration pattern and the sucking response over the feeding session.

A device for measuring infant feeding performance including a body portion, a respiration sensing device for receiving respiration and an integrated circuit disposed in the body portion and electrically connected to the respiration sensing device. The body portion has a first end for receiving a fluid, a second end for passing fluid to a feeding nipple, and a conduit in fluid communication with the first end and the second end. The respiration sensing device is mechanically coupled to the body portion and is mateable with the feeding nipple. The respiration sensing device is configured to generate a signal representing a variation in temperature or a change of airflow of the respiration during a feeding session. The integrated circuit is configured to receive the temperature signal or airflow signal and to determine a respiration pattern over the feeding session indicative of the infant feeding performance.

A system for measuring infant feeding performance including a fluid source for storing a comestible fluid, a feeding nipple, a body portion disposed between and coupled to the fluid source and to the feeding nipple, a respiration sensing device disposed between and coupled to the body portion and the feeding nipple, a pressure sensor disposed within the body portion and an electronics system. The electronics system is included within the body portion and is electrically connected to the pressure sensor and the respiration sensing device. The body portion includes a conduit in fluid communication with the fluid source and the feeding nipple. The respiration sensing device is configured to receive respiration and to generate a signal representing a variation in temperature or change of airflow of the respiration during a feeding session. The pressure sensor is configured to generate a signal representing a pressure of the fluid passing through the conduit during the feeding session. The electronics system is configured to receive the temperature or airflow signal and the pressure signal and to determine at least one of a respiration pattern and a sucking response indicative of the infant feeding performance.

### BRIEF DESCRIPTION OF THE DRAWINGS

The invention may be understood from the following detailed description when read in connection with the accompanying drawings. It is emphasized that, according to common practice, various features/elements of the drawings may not be drawn to scale. On the contrary, the dimensions of the various features/elements may be arbitrarily expanded or reduced for clarity. Moreover, in the drawings, common numerical references are used to represent like features/elements. Included in the drawings are the following figures:

FIG. 1 is a side view diagram of an exemplary system for measuring infant feeding performance, according to an embodiment of the present invention;

FIGS. 2A and 2B are perspective view diagrams of exemplary respiration sensors included in the system shown in FIG. 1, according to embodiments of the present invention;

FIGS. 3A and 3B are circuit diagrams of exemplary amplification circuits for the respiration sensor of the system shown in FIG. 1, according to embodiments of the present invention; and

FIGS. 4A and 4B are graphs of example respiration patterns as a function of time measured using the system shown in FIG. 1, according to embodiments of the present invention.

FIGS. 5A and 5B are perspective drawings of an example alternative respiration sensor.

### DETAILED DESCRIPTION OF THE INVENTION

Aspects of the present invention include an independent, hand-held device for measuring, recording, and optionally monitoring two or more feeding factors, including the respiration pattern and sucking response, related to the feeding performance of an infant, for example, during a feeding session. To measure the feeding factors, an exemplary device may include a respiration sensing device, a sucking response sensing device, embedded electronics and a computerized data processing system. The hand-held device may be used in conjunction with a nipple and a fluid reservoir holding a comestible fluid. An exemplary respiration sensing device includes a sensor for measuring a change in temperature or airflow in response to the inspiration and expiration of the infant during the feeding session. The respiration sensor is desirably placed directly on the hand-held device, proximate to the nipple and spaced apart from the infant's face. The change in temperature or airflow may correspond to a respiration pattern of the infant during the infant's inspiration and expiration of air. An exemplary sucking response sensing device may measure a fluid pressure within and a fluid flow through the sucking response sensing device during the feeding session. It is contemplated that the exemplary device may function independently of an external system for data collection, recording, and/or analysis.

A feeding session refers to a continuous period of time during which an infant (e.g., a human child from age birth to about one-year old) is provided with a feeding device, such as a baby bottle, and is encouraged to feed from the device. The feeding session may be a function of a predetermined period of time, a predetermined consumption volume, or both. A feeding performance of the infant may be determined during the feeding session. The feeding session also can be subdivided into two or more epochs to further quantify the feeding performance of an infant. Feeding sessions also can be subdivided in relation to one or more events or markers such as an instance of particular behavior demonstrated by the observed infant. Although the present invention is described with respect to human infants, it is contemplated that exemplary devices and systems of the present invention may also be used to measure the feeding performance of animal infants.

Feeding performance refers to an infant's innate or acquired capacity for orally feeding via a synthetic nipple, expressed as physical, physiological and/or behavioral responses during a feeding session. Feeding factors relate to one or more physical, physiological, and/or behavioral responses produced or exhibited by an infant while orally feeding or attempting to orally feed. Examples of feeding factors include respiration, sucking pressure, expression pressure, oxygen saturation level and swallowing.

As used herein, the term electronics relates to devices and systems that utilize the conductive or semiconductive flow of electricity and may include electronic sensors, microprocessors, microcontrollers, printed circuit boards, signal amplifiers, analog to digital converters, microelectromechanical systems, memory chips, electronic displays, microphones, electrical relays, switches, transceivers, and the like. Transceivers may include wired transceivers and wireless transceivers (e.g., Bluetooth devices).

As used herein, the term sensor relates to a device which measures a physical quantity, and converts it into an electrical signal, such as a digital signal via an analog-to-digital converter, which may be read by an instrument, for example, a microprocessor or microcontroller. Examples of sensors include photoelectric elements, piezoresistors, piezoelectrics, pyroelectrics, thermistors and pulse oximeters utilizing infrared sources, microphones for recording comments during a feeding session, as well as fiber optic elements for measuring temperature, pressure, and strain.

As used herein, the term embedded relates to being included within the confines of an article, including the article's interior and surface, or positioned on the outer surface of the article. Embedded electronics are contrasted with remote or external electronics which are outside the confines of the article, regardless of whether the electronics are in electronic communication with the article via a wired or wireless data transfer system. Embedded electronics may be included permanently within the confines of the article or may be temporarily removable from the confines of the article.

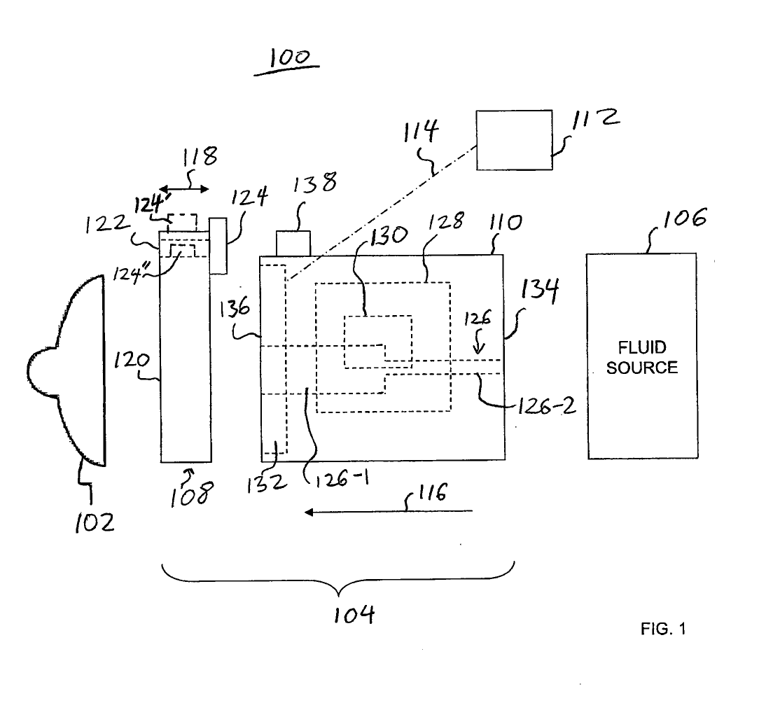

Referring to FIG. 1, a side view diagram of an exemplary system 100 for measuring infant feeding performance is shown. System 100 includes nipple 102, device 104 for measuring at least two feeding factors and fluid source 106. System 100 may also record and/or monitor the feeding factors as a function of time. In an exemplary embodiment, device 104 includes respective sensors (described further below) for simultaneously measuring the respiration pattern of an infant and the pressure and flow of a comestible fluid as a function of time.

Fluid source 106 may include any suitable reservoir for holding a comestible fluid, such as a conventional baby bottle. The comestible fluid may include, for example, an infant formula, expressed breast milk, pediatric electrolyte solution or water. The baby bottle may be of any conventional capacity, including, but not limited to, 4 oz. bottles and 8 oz. bottles.

Device 104 may be fastened to fluid source 106 and to nipple 102 by any suitable fasteners. For example, fluid source 106 and nipple 102 may be releasably fastened to device 104 via respective fittings, such as threaded fittings. According to an exemplary embodiment, device 104 may include threaded fittings that are functionally equivalent to the fittings of conventional infant nipple collars and conventional baby bottles. Using functionally equivalent threaded fittings may allow device 104 to be easily fastened to conventional baby bottles and nipple collars without the need for additional mating adaptors.

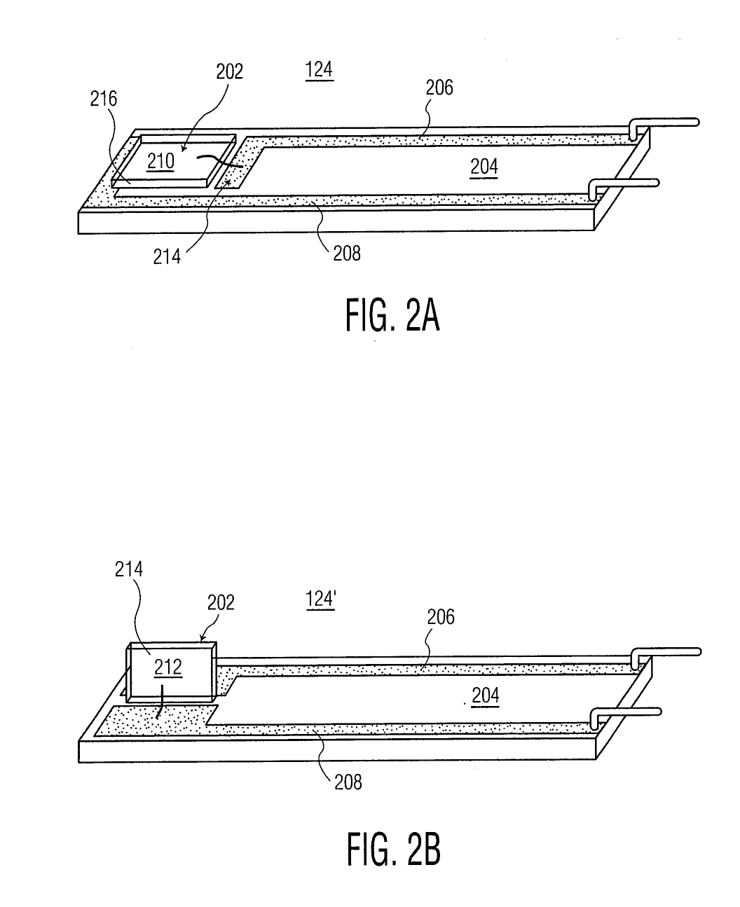

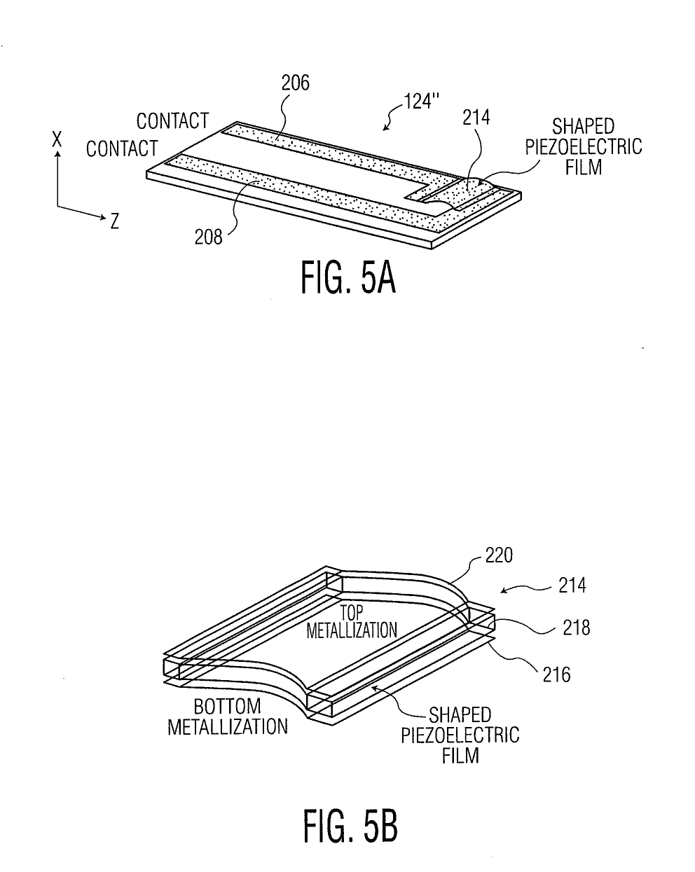

Device 104 may include a respiration sensing device 108 and body portion 110. Body portion 110 includes a fluid inlet side 134 coupled to fluid source 106 and a fluid outlet side 136, coupled to nipple 102 via collar 120 of respiration sensing device 108, and defining a fluid flow direction 116. First and second example respiration sensors 124 and 124′, described below with reference to FIGS. 2A and 2B, measure a change in temperature with respiration corresponding to a respiration pattern of the infant during the feeding session. A second example respiration sensor 124″, described below with reference to FIGS. 5A and 5B, measures respiration by measuring force applied to a piezoelectric element.

Body portion 110 measures a sucking response of the infant during the feeding session. The respiration pattern is associated with the maturational and functional status of infant feeding. For example, the respiration pattern may be used to detect abnormalities such as apnea, which may disrupt the feeding pattern, or aspiration. The respiration pattern and the sucking response may be used in combination to determine the feeding performance of the infant. For example, a rhythmic breathing, tightly coupled to the sucking pattern, may indicate a mature feeding ability and is typical of healthy full-term infants.

Respiration sensing device 108 may include collar 120 and respiration sensor 124. Collar 120 may be used to secure nipple 102 to body portion 110 and to allow comestible fluid to pass through fluid outlet side 136 of body portion 110 to nipple 102. Collar 106 may also include an air flow channel 122 to direct inspiration and expiration of the infant's breath, represented by double arrow 118, to respiration sensor 124. Respiration sensor 124 may be electrically coupled to an integrated circuit 132 of body portion 110 (described further below). Although respiration sensor 124 is illustrated as being positioned against air flow channel 122, respiration sensor 124 may be spaced apart from air flow channel 122, such that air channel 122 is open at both ends. According to other embodiments, the respiration sensor may be positioned on top of collar 120 or may be positioned within air flow channel 122, as shown by respective respiration sensors 124′, 124″.

According to another aspect of the invention, respiration sensor 124 may also include a pyroelectric anemometer. The pyroelectric anemometer may be used to monitor a flow rate of the respiration. An example pyroelectric anemometer is disclosed in U.S. Pat. No. 4,332,157 entitled PYROELECTRIC ANEMOMETER, the contents of which are incorporated herein.

In an exemplary embodiment, respiration sensor 124 may be used to monitor a respiration pattern of the infant during the feeding session, by measuring a variation in temperature or airflow with inspiration and expiration of the infant's breath across respiration sensor 124. Respiration sensor 124 is described further below with respect to FIGS. 2-4B. A signal generated by respiration sensor 124 may be received by integrated circuit 132 where it may be digitally converted into and, optionally, stored as a respiration pattern.

As shown in FIG. 1, respiration sensor 124 is directly mechanically coupled to body portion 110 (and nipple 102) and is positioned such that it is not in contact with the infant. Respiration sensor 124 may be positioned by any suitable distance from the infant's face such that it is capable of receiving a temperature or airflow change indicative of a respiration pattern. In an exemplary embodiment, respiration sensor is between about 1 cm-8 cm, more preferably 1 cm-4 cm, from the infant's face. Because respiration sensor 124 is not in contact with the infant, respiration sensor 124 may be less irritative and disruptive as compared with conventional nasal hot-thermistors, which are typically used to monitor respiration.

Body portion 110 includes hollow conduit 126 in fluid communication with respective fluid inlet and outlet sides 134, 136 and a pressure sensor assembly 128. In general, body portion 110 includes a sucking response sensing device which includes pressure sensor assembly 128 and conduit 126. Pressure sensor assembly 128 includes pressure sensor 130 for monitoring pressure and/or fluid flow in conduit 126. Body portion 110 also includes integrated circuit 132 at least partially disposed within body portion 110. Integrated circuit 132 may record and optionally monitor the respiration pattern from respiration sensor 124 and the pressure and/or fluid flow from pressure sensor 130 during the feeding session.

Body portion 110 houses and physically protects the embedded electronics (i.e., pressure sensor assembly 128 and integrated circuit 132) of device 104. For convenience and functionality, body portion 110 may be sized in proportion to a baby bottle so that the baby bottle and attached device 104 may be easily held by a nurse or other individual while the infant is feeding from the bottle. According to an exemplary embodiment, the volumetric size of body portion 110 is no greater than the volumetric size of a conventional 4 oz. baby bottle.

Collar 120 and body portion 110 may be constructed of any material or combination of materials suitable for infant feeding, clinical, and/or culinary applications. According to an exemplary embodiment, the materials of construction may include a comestible-grade metal or plastic. In some embodiments, body portion 110 may be constructed of a shapeable, more preferably moldable, thermoplastic resin such as polyethylene and the like. Although not shown in FIG. 1, device 104 may include a housing to cover body portion 110.

Pressure sensor assembly 128 is configured to measure the sucking pressure that is applied to the tip of nipple 102 by the infant's sucking action, based on the pressure of the fluid passing through conduit 126. An exemplary pressure sensor assembly 128 is disclosed in PCT application US2009/041782 entitled DEVICE FOR MEASURING INFANT FEEDING PERFORMANCE, the contents of which are incorporated herein. In general, when fluid flow decreases, a higher pressure is measured and, when fluid flow increases, a lower pressure is measured.

A signal generated by pressure sensor 130 is received by integrated circuit 132 where it may be digitally converted into, and, optionally stored as, pressure and/or flow data. According to an exemplary embodiment, the signal generated by pressure sensor 130 may be digitally sampled at an appropriate rate for further analysis either by integrated circuit 132 or when downloaded to a remote computer. In an exemplary embodiment, the signal generated by pressure sensor 130 may be sampled at a rate of about 100 Hz to about 1000 Hz. According to an exemplary embodiment, data from respiration sensor 124 may be sampled at the same sampling rate as the data from pressure sensor 130. As an example, respiration data (from respiration sensor 124) may be sampled at time N and pressure data (from pressure sensor 130) may be sampled at time N+2.5 ms, where N is the elapsed time in 5 ms units.

Pressure sensor 130 may include a sensor element, a meter for measuring electrical potential, current, resistance, or similar electrical effect produced by the sensor element, and a housing. Examples of sensor elements include strain gauges, piezoresistors, and the like. In an exemplary embodiment, a change in resistance produced by a piezoresistor with applied mechanical stress may be measured using a built-in Wheatstone bridge or similar circuit. That is, the piezoresistor produces a change in resistance that is measured by the Wheatstone bridge. The Wheatstone bridge, in turn, produces an output voltage signal that may be converted into a digital signal for further processing. Pressure sensor 130 may also include other electrical components, such as an amplifier and the like. An example of a commercially available pressure sensor includes a Freescale MPX2300 manufactured by Motorola, Inc. of Schaumburg, Ill., USA. According to an exemplary embodiment, suitable pressure sensors 130 may include those capable of measuring pressure over a range of about 10 mg Hg to about 300 mg Hg, with an accuracy of about ±1%.

In an exemplary embodiment, pressure sensor assembly 128 is positioned along the axis of the hollow conduit 126 so that hollow conduit 126 passes through the assembly. Pressure sensor 130 may be disposed so as to contact sections 126-1, 126-2 of conduit 126. Section 126-2 has a cross-sectional area smaller than the cross-sectional area of section 126-1. Because the fluid flowing through the hollow conduit 126 encounters sections 126-1, 126-2 of different cross-sectional areas, a rate of fluid flow can be determined by measuring the pressure inside capillary 126, using atmospheric pressure as a reference pressure. The fluid flow rate may then be determined from the measured pressure, for example by applying Bernoulli's principle, which describes an inverse relationship between fluid pressure and a fluid flow rate. For example, that as the fluid pressure decreases, the fluid flow rate increases. The fluid pressure and fluid flow rate may be used to determine one or more feeding factors.

Integrated circuit 132 may include one or more microcontrollers and/or microprocessors for generating and processing the data from respiration sensor 124 and pressure sensor 130. Integrated circuit 132 may also include one or more memory chips, such as flash memory, for storing the data from respiration sensor 124 and/or pressure sensor 130. Integrated circuit 132 may also include at least one of an analog-to-digital converter, a wireless transceiver or a wired transceiver.

Integrated circuit 132 may be embedded in body portion 110 and may be fixed by a friction fitting, adhesive, mechanical faster, or related device. Integrated circuit 132 may be in operative contact with a surface of body portion 110 to provide or receive information to or from an operator. For example, the operator may indicate initiation of a feeding session to integrated circuit 132. As another example, integrated circuit 132 may provide an indication of recording of a respiration pattern and/or a sucking response. In some embodiments, a microcontroller of integrated circuit 132 may be programmable and may analyze the data, compute trends based upon the data, and/or generate a medically relevant assessment score for the patient as a function of the respiration pattern, the pressure data and the flow data. For example, time characteristics of both the sucking response and the respiration pattern may be measured to determine any correlation between these two measurements over the feeding session.

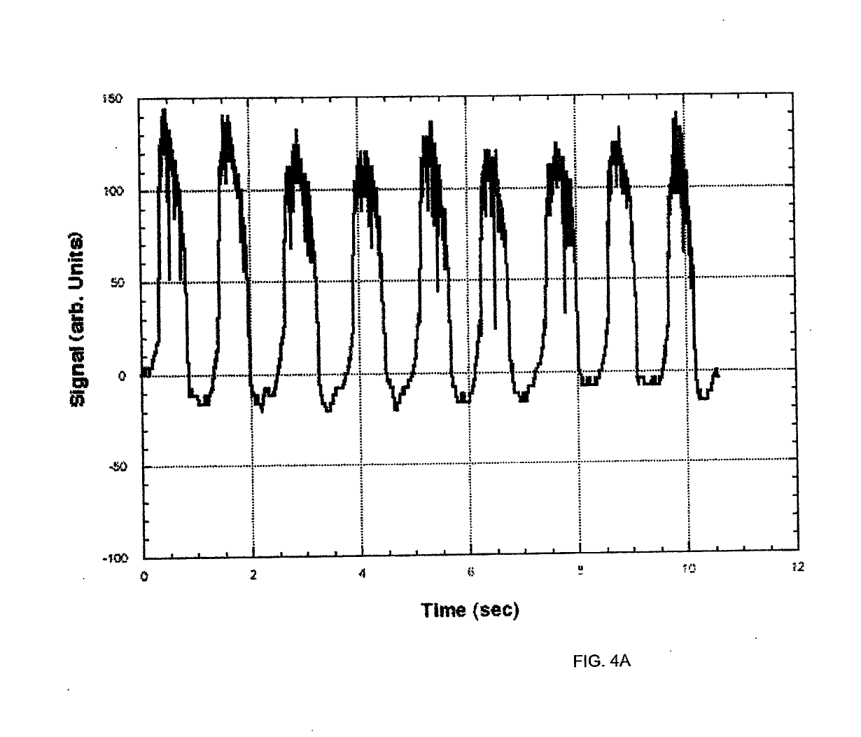

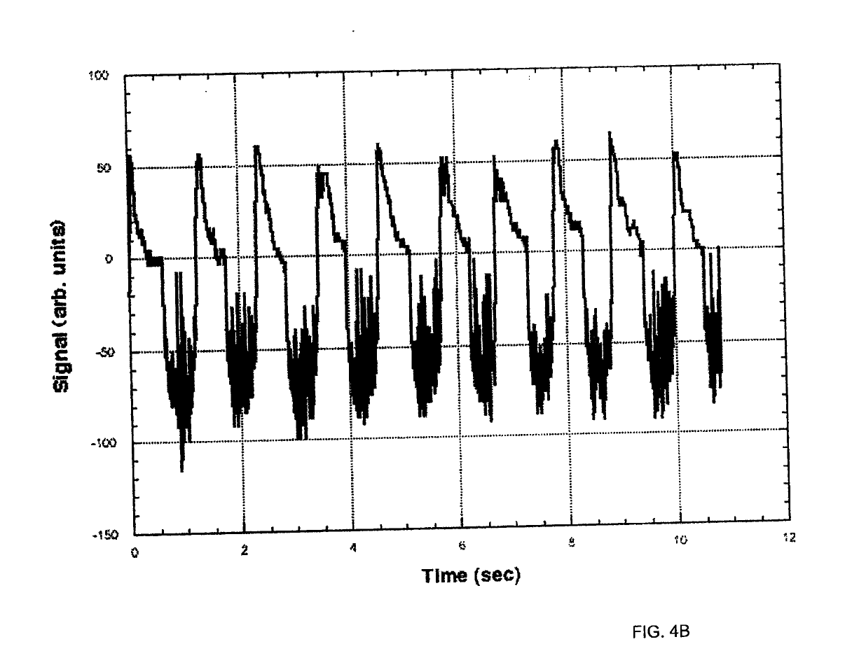

Device 104 may also include a battery (not shown) and/or a user interface assembly 138. User interface 138 may include one or more features such as buttons, indicators, an electronic display, a touch screen, a microphone, a speaker, a bar-code scanner and the like. User interface 138 may display, for example, real-time computational results from the sucking response data and/or the respiration data. These optional components may allow the operator to setup, control and monitor the device, provide feedback, including alarms, to the operator, and/or allow the operator to input additional data such as voice recordings or event markers correlating to observations of the operator during a feeding session. An indicator may also be used to provide feedback to the operator, such as to provide an indication of a peak pressure of the fluid within device 104. A display may display simplified waveforms such as those shown in FIGS. 4A and 4B or other indicators of respiration and feeding performance to allow the caregiver to determine if there are problems with the device (e.g., obstacles in the fluid conduit or placement of the respiration sensor) or problems with the infant (e.g., partially obstructed nasal passages).

System 100 may also include one or more optional sensors 112 for measuring one or more additional feeding factors, and an optional data communication link 114 for transmitting data between and/or receiving data from the optional sensor 112 or an external data recording/data processing device (not shown). Communications link 114 may be a wired or wireless link. Sensor 112 may be embedded in body portion 110, embedded in nipple 102, embedded in collar 120 or may be external to system 100. External sensor 112 which is not embedded in device 104, may be in data communication with device 104 via a wireless data transfer system and/or via a wired data transfer connection. The data produced via these sensors may be transmitted to a remote recording device and/or may be stored in the embedded memory of device 104 for subsequent retrieval.

Examples of other sensors 112, that may be provided with system 100 include a pulse oximeter and/or infrared (IR) optical sensor mounted in feeding nipple 102. Such sensors may be used to measure the infant's blood oxygen saturation and CO₂ elimination, respectively, during a feeding session. According to another embodiment, a chemical sensor may be attached to the surface of feeding nipple 102 to detect and monitor trace chemicals and ions in the infants saliva. According to yet another embodiment, a piezoelectric impulse sensor may be attached to nipple 102 to measure an infant's swallowing capacity. As the neonate swallows, the tongue blocks the front of the mouth, compressing nipple 102 in the process. The resulting signal may provide useful information to the caregivers about the feeding process of the neonate.

Referring to FIGS. 2A and 2B, exemplary respiration sensors 124, 124′ are shown. Respiration sensor 124 includes pyroelectric element 202 electrically connected to respective first and second electrodes 206, 208 on circuit board 204. Pyroelectric element 202 is desirably sensitive to the temperature difference between an infant's inhaled and exhaled air at a predetermined distance from the infant's face.

In certain classes of crystals, an electric polarization along a particular crystallographic axis may not vanish. Changing the temperature in such crystals produces a change in the electric polarization, known as the pyroelectric effect. Accordingly, if pyroelectric element 202 is heated, a surface charge is typically formed, which may be measured by appropriately connected electrodes, such as electrodes 206, 208.

In general, as the temperature of pyroelectric element 202 changes, the generated surface charge may induce a current flow in an external circuit connected to element 202. When element 202 is heated, current may flow in a first direction. When element 202 is cooled, current may flow in a second direction opposite the first direction. The variation in current flow with temperature may be used by element 202 to monitor an infant's respiration, where exhaled air is typically warmer than inhaled air.

In operation, pyroelectric element 202 may generate a time-varying current that is directly proportional to the heat uptake and loss from the air moving over pyroelectric element 202 (as indicated by double arrow 118 in FIG. 1). Because the change in current flow occurs directly from the change in temperature, and because the infant's respiration cycle constantly changes with inspiration, pyroelectric element 202 may be directly driven by the infant's respiration over the feeding session. Accordingly the infant's respiration may drive element 202 without using any additional electrical current.

Referring to FIGS. 4A and 4B, examples of respiration patterns measured as a function of time measured using the respiration sensor 124 (with the configuration as shown in FIG. 2A) are shown. Although pyroelectric element 202 may not measure an absolute air flow volume from the infant's respiration, pyroelectric element 202 does measure a time-varying current indicative of a respiration pattern. The respiration pattern corresponds to the inspiration and expiration of the infant over time. The respiration pattern may be analyzed for breath-events and characteristics to determine the infant's feeding performance over the feeding session. For example, the amplitude of flow-related excursion and the waveform shape (e.g., the rate of rise and the shape of the peak) may be determined from the respiration pattern.

In an exemplary embodiment, pyroelectric element 202 may be formed from lithium tantalate (LiTaO₃). It is understood other pyroelectric materials may be used, including, but not limited to polyvinylidene fluoride (PVDF 2), strontium barium niobate, plastic pyroelectrics and other crystalline pyroelectrics.

In an exemplary embodiment, pyroelectric element 202 may be electrically shielded. For example, pyroelectric element 202 may be placed in proximity to a conducting material that is electrically grounded. For example, the conducting material may be a metallic film deposited inside of air flow channel 122.

As discussed above, because the infant's respiration across element 202 induces a current flow, respiration sensor 124 does not use an additional power source. In addition, respiration sensor 124 detects a respiration pattern, not an absolute volume of air flow, based on changes in temperature due to the infant's inspiration and expiration. Because respiration sensor 124 detects a respiration pattern, respiration sensor 124, in contrast to conventional hot-thermistor devices, does not include reference circuitry to adjust the output signal relative to changes in ambient temperature.

In FIG. 2A, a bottom surface 212 (shown in FIG. 2B) of pyroelectric element 202 is formed on circuit board 204. Bottom surface 212 is also directly electrically connected to second electrode 208. A top surface 210 of pyroelectric element 202 is electrically connected to first electrode 206 via connector 214. Respiration sensor 124′ (FIG. 2B) is the same as respiration sensor 124 (FIG. 2A) except that a side surface 216 (shown in FIG. 2A) of pyroelectric element 202 is formed on circuit board 204, with each of top surface 210 and bottom surface 212 electrically connected to respective first and second electrodes 206, 208 via connectors 214.

It is understood that any conductive material may be used for first and second electrodes 206, 208 and connectors 214. Examples of conductive material include, but are not limited to, aluminum, gold, nickel, nickel-chromium, silver, platinum, titanium, tungsten or copper. Circuit board 204 may include any suitable electrically insulating material, such as, for example, resin, ceramic or plastic materials. In an exemplary embodiment, circuit board 204 includes a two-sided copper-clad printed circuit board.

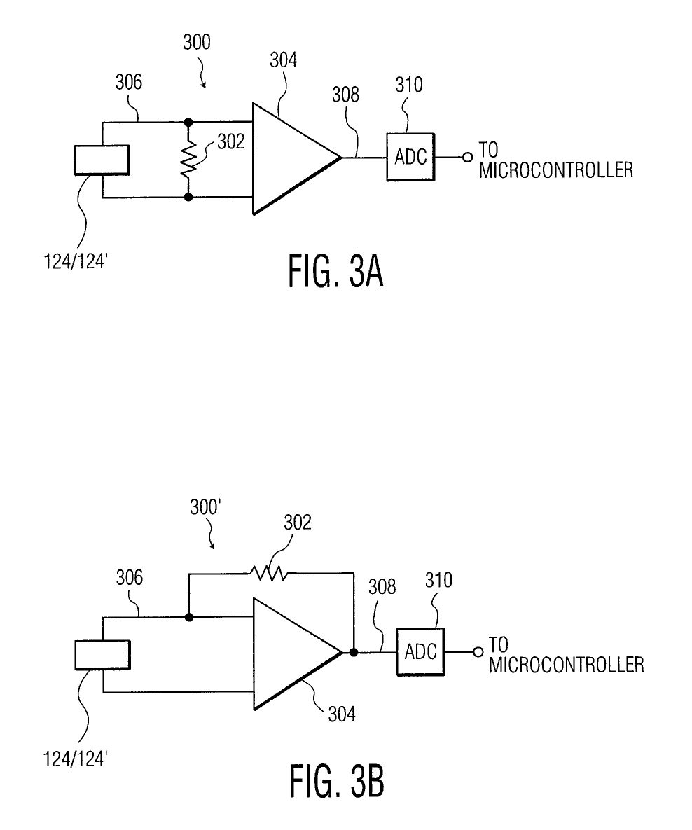

Referring to FIGS. 3A and 3B, exemplary amplification circuits 300, 300′ for respiration sensors 124, 124′ (FIGS. 2A and 2B) are shown. Amplification circuits 300, 300′ may be formed as part of integrated circuit 132 (FIG. 1) or may be formed on circuit board 204 (FIG. 2A). In FIG. 3A, amplification circuit 300 includes resistor 302 and amplifier 304 connected in parallel with respiration sensor 124. An electrical signal from pyroelectric element 202 (FIG. 2A) appears in the form of an electrical charge on electrodes 206, 208. Resistor 302 allows a discharge of the electrical charge, where the discharge produces an electrical voltage signal 306 over resistor 302. Amplifier 304 provides suitable amplification of voltage signal 306 to generate output signal 308. In general, the value of resistor 302 depends on the inherent gain of amplifier 304. Output signal 308 may be digitized by analog to digital converter (ADC) 310 and sampled and collected by a microcontroller of integrated circuit 132 (FIG. 1). Although ADC 310 is shown separate from the microcontroller, it is understood that ADC 310 may be part of the microcontroller.

As shown in FIG. 3B, amplifier 304 may be formed in parallel with respiration sensor 124, 124′ and resistor 302 may be formed as a feedback resistor between the output of amplifier 308 and the output of pyroelectric element 202 (FIG. 2A). In this configuration, circuit 300′ forms an current amplifier. In a current amplifier, the current produced by the discharge of the electrical charge is converted into an output voltage. Accordingly, output voltage signal 308 is proportional to the change of the electrical charge on pyroelectric element 202 (FIG. 2A).

In an exemplary embodiment, after amplification, the output signal 306 is less than or equal to about 2.5 V. In general, the voltage of output signal 306 depends upon the applied voltage (e.g., a battery used to power the embedded electronics). It is understood that a suitable pyroelectric element 202 (FIG. 2A) and amplification circuit components may be selected which allow detection of breath-events in the respiration pattern.

FIGS. 5A and 5B show an alternative example respiration sensor 124″. As shown in FIG. 5A, this alternative sensor has the same form factor as the sensors 124 and 124′, described above. It includes conductors 206 and 108 which are coupled, respectively, to the top and bottom electrodes of piezoelectric device 214. The device 214 includes a bottom conductor 216, a shaped piezoelectric element 218 and a top conductor 220. In the example sensor 124″, the piezoelectric element is a polyvinylidene difforide (PVDF) piezoelectric film.

In this example, the piezoelectric device 214 is shaped as an airfoil. Any air flowing over the airfoil induces a small lift force that strains the piezoelectric element 218 along the Z-axis, thereby inducing a charge in the direction of the X-axis. The induced charge varies in response to the force of the respiration and provides an electric current that may be amplified by the same amplification circuits 300 and 300′ (described above) that are used with the pyroelectric sensors 124 and 124′.

The piezoelectric sensor may be less expensive than the pyroelectric sensor as PVDF is relatively inexpensive compared to LiTaO₂. In addition, the piezoelectric sensor may be more rugged than the pyroelectric sensor. In addition, the metalized film is relatively easy to fabricate in different forms that respond to airflow induced by breathing. In this regard, although the sensor 124″ is described as being an airfoil, it is contemplated that other shapes may be used. For example, the piezoelectric sensor may be vertical having a face toward the infant such that the infant's respiration causes a slight deflection of the sensor, inducing the current. In addition, although the piezoelectric element is described as being formed from PVDF, it is contemplated that any piezoelectric material may be used. In general, any material having a cubic structure that lacks mirror symmetry in one direction may serve as a piezoelectric material. These materials may be plastic or ceramic.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. A method of measuring infant feeding performance comprising:
passing fluid to a feeding nipple through a conduit;
passing respiration across a respiration sensor mechanically coupled to the feeding nipple;
monitoring a pressure of the fluid passing through the conduit during a feeding session in the conduit;
monitoring the respiration across the respiration sensor during the feeding session; and
determining at least one of a sucking response from the monitored pressure or a respiration pattern from the monitored respiration over the feeding session indicative of the infant feeding performance.

2. The method according to claim 1, wherein the monitoring of the respiration includes monitoring a change in temperature during inspiration and expiration of the respiration over the feeding session, the change in temperature indicative of the respiration pattern.

3. The method according to claim 2, wherein the temperature of the respiration is monitored without contacting a source of the respiration.

4. The method according to claim 1, wherein the monitoring of the respiration includes monitoring a change in airflow during inspiration and expiration of the respiration over the feeding session, the change in airflow indicative of the respiration pattern.

5. The method according to claim 1, the passing of the respiration across the respiration sensor includes:
directing the respiration through a conduit to the respiration sensor.

6. The method according to claim 1, wherein the sucking response includes at least one of a fluid pressure response, a fluid flow response or a sucking pressure response.

7. The method according to claim 1, wherein the conduit includes a first section to receive the fluid and a second section to pass the fluid to the feeding nipple, the first section having a different cross-sectional area than the second section and monitoring the pressure of the fluid includes:
monitoring the pressure of the fluid passing through the conduit at a position in the conduit between the first section and the second section.

8. A device for measuring infant feeding performance comprising:
a body portion having a first end for receiving a fluid, a second end for passing fluid to a feeding nipple, and a conduit in fluid communication with the first end and the second end;
a respiration sensing device for receiving respiration, the respiration sensing device being mechanically coupled to the body portion, the respiration sensing device configured to generate a signal representing the respiration during a feeding session; and
an integrated circuit disposed in the body portion and electrically connected to the respiration sensing device, the integrated circuit configured to receive the respiration signal and to determine a respiration pattern over the feeding session indicative of the infant feeding performance.

9. The device according to claim 8, wherein the integrated circuit includes at least one of a microcontroller or a microprocessor.

10. The device according to claim 8, wherein the integrated circuit includes a wireless data transceiver or a wired data transceiver.

11. The device according to claim 8, further including a user interface integrated with the body portion and in electronic communication with the integrated circuit.

12. The device according to claim 11, wherein the user interface includes at least one of an input device, an indicator or a display.

13. The device according to claim 8, wherein the respiration sensing device includes:
a collar mateable with the feeding nipple and mechanically coupled to the body portion, and
a respiration sensor for receiving the respiration, the respiration sensor electrically connected to the integrated circuit and disposed on the collar, the respiration sensor generating the respiration signal.

14. The device according to claim 13, wherein the respiration sensor includes a pyroelectric element that is configured to measure a change in temperature between inspiration the expiration during the respiration.

15. The device according to claim 13, wherein the respiration sensor includes a piezoelectric element that is configured to measure a change in airflow during the respiration.

16. The device according to claim 15, wherein the piezoelectric element is configured as an airfoil and positioned in the collar such that the respiration passes over the airfoil.

17. The device according to claim 13, wherein the collar includes an air flow channel configured to direct the respiration to the respiration sensor.

18. The device according to claim 13, wherein the respiration sensor includes an amplification circuit for amplifying the respiration signal.

19. The device according to claim 13, wherein the integrated circuit includes an amplification circuit for amplifying the respiration signal.

20. The device according to claim 8, further comprising:
a pressure sensor disposed in the body portion and in contact with the fluid in the conduit, the pressure sensor configured to generate a signal representing a pressure of the fluid passing through the conduit during the feeding session,
wherein the integrated circuit is electrically connected to the pressure sensor, the integrated circuit being configured to receive the pressure signal and to determine a sucking response over the feeding session indicative of the infant feeding performance.

21. The device according to claim 20, wherein the integrated circuit includes a memory chip for storing at least one of the respiration pattern or the sucking response.

22. The device according to claim 20, wherein determination of the sucking response includes determining at least one of a fluid pressure response, a fluid flow response or a sucking pressure response.

23. A system for measuring infant feeding performance comprising:
a fluid source for storing a comestible fluid;
a feeding nipple;
a body portion disposed between and coupled to the fluid source and to the feeding nipple, the body portion including a conduit in fluid communication with the fluid source and the feeding nipple;
a respiration sensing device disposed between and coupled to the body portion and the feeding nipple, the respiration sensing device configured to receive respiration and to generate a respiration signal representing a variation of the respiration during a feeding session;
a pressure sensor disposed within the body portion and configured to generate a signal representing a pressure of the fluid passing through the conduit during the feeding session; and
an electronics system included within the body portion and electrically connected to the pressure sensor and the respiration sensing device, the electronics system configured to receive the respiration signal and the pressure signal and to determine at least one of a respiration pattern and a sucking response indicative of the infant feeding performance.

24. The system according to claim 23, further including a user interface integrated with the body portion and in electronic communication with the electronics system.

25. The system according to claim 23, wherein the electronics system includes a memory chip for storing at least one of the respiration pattern or the sucking response.

26. The system according to claim 23, wherein the electronics system includes a wireless data transceiver or a wired data transceiver.

27. The system according to claim 23, wherein the respiration sensing device includes:
a collar disposed between and coupled to the body portion and the feeding nipple, and
a respiration sensor for receiving the respiration, the respiration sensor electrically connected to the electronics system and disposed on the collar, the respiration sensor generating the respiration signal.

28. The system according to claim 27, wherein the respiration sensor includes a pyroelectric element that is configured to measure a change in temperature between inspiration the expiration during the respiration.

29. The device according to claim 27, wherein the respiration sensor includes a piezoelectric element that is configured to measure a change in airflow during the respiration.

30. The device according to claim 27, wherein the piezoelectric element is configured as an airfoil and positioned in the collar such that the respiration passes over the airfoil.

31. The system according to claim 27, wherein the collar includes an air flow channel configured to direct the respiration to the respiration sensor.
